(19) United States
(12) Patent Application Publication
(10) Pub. No.: US 2020/0008998 A1
(43) Pub. Date: Jan. 9, 2020

(54) MANDIBULAR/HEAD POSITIONER FOR PATIENTS UNDERGOING ANESTHESIA

(71) Applicant: Anand MORANKAR, New York, NY (US)

(72) Inventor: Anand MORANKAR, New York, NY (US)

(21) Appl. No.: 16/483,068

(22) PCT Filed: Feb. 2, 2018
(86) PCT No.: PCT/US2018/016569

§ 371 Date: Aug. 2, 2019

Related U.S. Application Data
(60) Provisional application No. 62/453,598, filed on Feb. 2, 2017.

(51) Int. Cl. A61G 13/12; A61M 16/06; A61G 13/10

(52) U.S. Cl. CPC A61G 13/121; A61M 16/0694; A61M 2210/06; A61M 2209/082; A61M 2205/584; A61G 13/101

(57) ABSTRACT

Systems and methods for positioning a patient's head and neck during surgery and/or various medical procedures. A positioner for holding a patient's head and/or neck in static orientation throughout a medical procedure that is capable of easy readjustment as required.

### CROSS REFERENCE TO RELATED APPLICATIONS

This application claims the benefit of Provisional U.S. Patent Application 62/453,598 filed Feb. 2, 2017 entitled MANDIBULAR/HEAD POSITIONER FOR PATIENTS UNDERGOING ANESTHESIA. The contents of this application are incorporated by reference in its entirety.

### FIELD OF THE INVENTION

The invention relates to an improved mandibular/head positioner device used during surgery and/or various medical procedures. More specifically, the present inventive method and apparatus relates to a mandibular/head positioner for holding and positioning a patient's mandible and/or head to optimize a patient's airway throughout a medical procedure. The mandibular/head positioner device is capable of easy readjustment as required.

### BACKGROUND OF THE INVENTION

Modern anesthesia dates to the early-to-mid 1800s. Most surgical procedures performed were usually “ambulatory” in that they were performed in private facilities or even in private homes as few hospitals existed away from military or academic settings. As the use of anesthetics during surgical procedures increased and became much more sophisticated and complex, both mask and endotracheal intubation became necessary as muscle relaxing medications were introduced which either incidentally or purposefully suppressed or halted patient respiration.

During medical procedures and surgeries requiring mild-to-moderate or even deep sedation; predominately, a conscious patient is placed supine before and/or during administration. Next, medical anesthetic gases and/or hypodermic intravenous formulations are administered. At this stage, the practitioner has mere minutes to intubate the non-breathing patient to avoid potentially fatal hypoxia. Typically, a member of the surgical team will intubate the patient in a supine “sniffing position.” A member of the surgical team will optimize the airway passageway using the “sniffing position” or a similar position.

Surgical positioning must ensure and maintain patient safety while allowing required access to a surgical site or sites. Paramount when selecting and adjusting a surgical position or series of positions is patient safety and good surgical outcome. Often a patient is placed in extremely unnatural positions for extended periods to gain access to a surgical site. Maintaining such positions becomes challenging when patients are in a relaxed state (a.k.a. an “anesthetized state”).

In addition, other considerations include relevant physical and physiological factors, such as patient body alignment, circulation, respiratory constraints, and the overall musculatory. Other variable factors include overall patient size, age, weight, physical condition, gender, and the like. The type and effectiveness of anesthesia also impact surgical positioning decisions, as well as the required surgical position effecting the type of anesthesia used.

Depending upon the complexity of a surgery or set of surgical procedures, in many instances a patient may be repositioned once or several times over several hours.

Modern surgical positions include, but are not limited to:

Supine Position: the most common of the surgical positions, where the patient lies with back flat on operating table or bed.

Trendelenburg Position: same as supine but the upper torso is lowered and legs are raised.

Reverse Trendelenburg Position: same as supine but upper torso is raised and legs are lowered.

Fracture Table Position: primarily for hip surgery, the upper torso is in supine position with unaffected leg raised, the affected leg extended with no lower support and strapped at the ankle with cushioning at the groin to keep pressure on the leg and hip.

Lithotomy Position: typically used for gynecological, anal, and urological procedures; upper torso is supine position, legs are raised and secured, arms are extended.

Fowler's Position: patient in supine position; upper torso is raised to a 90-degree position.

Semi-Fowlers Position: lower torso is supine and the upper torso bent at a nearly 85-degree position; patient's head secured by a restraint.

Prone Position: patient lies with stomach on the table or bed, in some instances, the abdomen is raised above table.

Jackknife or Kraske Position: patient's abdomen flat on the bed, bed scissored so hip is lifted with legs and head low.

Knee-Chest Position: like the Jackknife except legs are bent 90 degrees at the knee.

Lateral or Side-Laying Position: —lying position, like the Jackknife except the patient is on their side—including the Lateral-Chest and Lateral-Kidney positions.

Lloyd-Davies Position: common position surgery involving the pelvis and lower abdomen.

Kidney Position: much like the lateral position except the patient's abdomen is placed over a lift in or on the operating table that bends the body to allow access to the retroperitoneal space, at times a kidney rest is placed under the patient at the point of lift.

Sims' Position: is a variation of the left lateral position, the patient may be awake and help positioning, rolling to the side keeping the leg straight and bending the other—primarily used for colonoscopies, and trans-anal procedures.

All of these positions may in turn impact the head/neck position and consequently the airway passageway in combination with the effects of gravity.

As can be appreciated, depending upon the positioning required during surgery, being placed in such positions, and moved from position to position (especially when the surgery being performed involves the head and neck themselves), makes it difficult to maintain optimal airway positioning in a patient. This may and can be difficult, and in some instances, may be extremely difficult depending upon the positioning required.

Many times, a performing anesthesiologist, nurse-anesthetist, and/or anesthesia technician is required to have more than one pair of hands to keep up with the demands of optimizing airway positioning in order to optimize and ensure adequate oxygenation and ventilation.

Known successful intubation typically uses direct visual or video laryngoscopy, and is contingent upon alignment of the oral, pharyngeal and laryngeal axes in what is commonly called the “sniffing position.” In this position, a patient's head is slightly extended and the occiput is elevated approximately 5-7 cm in adults, and less in pediatric patients. Often, positioning the patient in this manner is enough to obtain a reasonable “percentage of glottic opening”, or POGO score, which allows identification of the usual laryngeal landmarks.

However, optimal airway positioning can be compromised due to a multiplicity of factors. Distortion (trauma, infection, neoplasm, edema etc.), disproportion (tongue/pharynx) or body habitus (particularly in obese patients), all can compromise or inhibit landmark recognition and make the sniffing position suboptimal or even at times inadequate or impossible.

The laryngoscopist may at times compensate for limited laryngeal exposure by lifting the patient's head off the bed or table using the laryngoscope itself. The average adult human head weighs between 8 to 10 lbs and, in some obese patients, lifting of the head and shoulders may be almost impossible.

However, medical literature has shown that laryngeal exposure can be improved with less required force by increasing head elevation and neck flexion. Without a mechanical device to enable this, support must be provided and maintained manually by a member of the surgical staff, or by placing “rolled up” surgical linens or other objects under the head, neck, and/or shoulders of the patient. To date, virtually no method or system has been developed to optimize patient head positioning when a difficult airway is encountered, or for use during or throughout a surgical or medical procedure. Furthermore, no method ensures perfect intubating conditions or glottic visualization.

Exemplary systems related to positioning a patient's head during surgical procedures include U.S. Pat. No. 4,259,757 issued to Watson and entitled “Support Cushion”, disclosing a cushion for medical use to support a patient's head and neck that can be utilized to achieve the sniffing position of the patient's head and torso to facilitate endotracheal intubations.

U.S. Pat. No. 5,048,136 discloses an infant support for airway management which aligns the oropharyngeal, laryngeal and tracheal axes. This support is in the form of a cushion with cut-outs which receive the head and torso of the infant. However, this mat is not adjustable in any way and lacks a highly desired repositioning capability or means.

Adjustable head and torso supports are known for example as shown in U.S. Pat. No. 5,528,783 issued to Kunz et al. which discloses an inflatable head and torso support which is adjustable whereby an air bladder can be fully inflated, partially inflated, or fully deflated as desired to incline the head or the head and torso. Inflation is controlled by valves that are in turn actuated by switches located on the edge of a sheet of material positioned under the torso of the user and attached to the support. The support is wedge-shaped and contains only one bladder. Therefore, it is incapable of individually elevating the head and torso portions of the user's body independently and therefore would not be appropriate as an ideal tracheal intubation body positioning support. However, as limited by configuration and design, Kunz is incapable of placing a patient in the supine “sniffing position.”

U.S. Pat. No. 5,632,283 discloses a chin support to hold an anesthetized patient's head during surgery so that the unconscious patient's airway is held open, including a pylon attached to and extends up from the side of an operating table. A tether attaches to the top of the pylon and extends around the patient's chin to keep the patient's head tilted during surgery. The pylon is held in place against the operating table by the tension on the tether when the chin support is in use. However, Kunz provides a single axis of support to the patient and that support only to the patient's chin, practically identical to known orthopedic systems for cervical and/or neck traction.

Other cited art includes U.S. Pat. Nos. 9,289,344, 8,601,623, 8,539,623, 8,001,636, 7,716,763, 7,383,599, 7,213,596, 7,127,758, 6,935,340, 6,672,308, 6,622,727, 6,490,737, 5,632,283, 5,524,639, 5,474,056, 5,410,769, 5,269,035, 5,184,365, 5,076,269, 4,844,061, 4,058,112, and RE34086; U.S. Patent Application Publication 20130333708; and foreign patents CN2577820Y, WO2010040120, CN201743662U.

However, none of the cited art provides a safe, quick and easy apparatus that provides precise maintenance of any one of a variety of positions.

### SUMMARY OF THE INVENTION

To improve upon the prior art, it is an object of the invention to provide an improved anesthesiologist's operating room device. In is an object of the invention to provide a device to assist in intubation and to be effective in fiberoptic bronchoscope (FOB) assisted intubation.

Commonly in the operating room (OR), a two-handed anesthesiologist needs a “third-hand” to hold either an object or to hold the patient in a particular position. With regards, to patient positioning, a particular posture may have to be intermittently readjusted or, in some cases, maintained for the duration of the procedure.

It is an object of the invention to provide a device that will allow for safe, precise, quick, easy, and consistent maintenance of any one of a variety of positions of a patient during a medical and/or surgical procedure.

It is another object of the invention for the mandibular device to maintain optimal airway positioning and to maintain head/neck position for the same and/or for differing surgical positioning.

It is another object of the invention to provide a device that allows for easy, quick, precise and possibly continuous, positioning of a patient's mandible head/neck during a medical and/or surgical procedure.

It is another object of the invention to provide a patient positioning device for supporting a patient's head, jaw, chin, or neck during a medical and/or surgical procedure.

It is another object of the invention to provide an easily adjustable device that can act as an anesthesiologist's “third hand” during a medical and/or surgical procedure.

It is another object of the invention to provide an adjustable device that supports a patient's chin and holds a patient's jaw in place in either a relaxed or forward compressed position during a medical and/or surgical procedure.

It another object of the invention to provide an adjustable device that will support a patient's chin at a mandibular angle to prevent the backwards motion of the mandible (“retrognathia”).

It another object of the invention to provide an adjustable device that will support a patient's chin and will create “prognathia”, which is the pulling of the jaw forward with some amount of force.

These and other objects of the invention are achieved by providing a patient positioning device for supporting a patient's head, jaw, chin, or neck during a medical procedure, the device comprising an upper support member comprising: a chin support member, a first jaw support member, and a second jaw support member, wherein upper support member includes an upper support member housing for the chin support member, and wherein the first jaw support member and second jaw support member are attached to the housing at one end thereof; an extension arm having a first end and a second end, said extension arm connected to said upper support member at the first end, wherein the extension arm has at least two degrees of freedom; an attachment member, the attachment member connected to said extension arm at the second end thereof, said attachment member configured to be attached at the other end to a hospital bed, rail or operating room stand.

In certain embodiments, the first jaw support and the second jaw support each includes a hand element.

In certain embodiments, the hand elements of each of the first and second jaw support members are configured to be adjustable with respect to the distance from one another and from the chin support member.

In certain embodiments, the hand elements are configured to hold a patient's chin at a mandibular angle to prevent the backwards motion of the mandible (“retrognathia”). In certain embodiments, this is achieved during medical or surgical procedure where a patient is sedated or receives anesthesia.

In certain embodiments, the hand elements are configured to create “prognathia”, which is the pulling of the jaw forward with some amount of force. In certain embodiments, the prognathia is required during medical or surgical procedure when a patient is sedated or receives anesthesia.

In certain embodiments, the hand elements are configured to exert a pressure on the patient's chin, wherein the patient's jaw is configured to be held in place in a forward position by the hand elements.

In certain embodiments, the positioning device includes a rotary joint connecting the attachment member to the extension arm and the extension arm to the upper support member.

In certain embodiments, the first jaw support member and the second jaw support are flexible and are configured to rotate with respect to the chin support member.

In certain embodiments, the first jaw support member includes a first upper arm member and a first lower arm member, and wherein the second jaw support member includes a second upper arm member and a second lower arm member, wherein the first lower arm member and the second lower arm member are each connected to the upper support member housing.

In certain embodiments, the first lower arm member and the second lower arm member are curved and have a C-shape orientation.

In certain embodiments, the first upper arm member and the second upper arm member are rods, wherein the first upper arm member is connected to the first lower arm member via a ball and socket joint, and wherein the second upper arm member is connected to the second lower arm member via a ball and socket joint. In certain embodiments, the rods have a cylindrical orientation.

In certain embodiments, the hand elements are configured to be adjusted along the length rods of the first upper arm member and second upper arm member respectively.

In certain embodiments, the chin support member includes a platform for supporting the chin of a patient, wherein the platform is wide enough to support a patient's chin having various sizes. In certain embodiments, the patient may be an adult, obese adult, child or infant.

In certain embodiments, the extension member comprises a first extension member and a second extension member that are connected via a first rotary joint.

In certain embodiments, the first extension member and the second extension member are configured to provide full articulation when providing adequate rigidity and support in order to place and hold a patient in a desired position.

In certain embodiments, the patient positioning device includes a pressure force sensor embedded in the chin support member.

In certain embodiments, the patient positioning device includes a display embedded within the positioning device, the display showing the colors red, yellow and green to associate different locking positions of the device. In certain embodiments, the color red means that the device is in a locked position, the color yellow means the device is a partially locked position, and the color green means the device is an unlocked position such that it can be manipulated or moved.

In certain embodiments, the attachment member is a C-clamp. In certain embodiments, the attachment member is a fastening means and/or tensioning device to tension and/or attach the patient positioning device to a support element, such as a hospital table or surgical support element.

In certain embodiments, the extension member is configured to be locked during support of the patient and unlocked during manipulation of the positioning device prior to positioning the patient.

In certain embodiments, the positioning device is configured to be intermittently readjusted during the medical procedure.

In certain embodiments, the positioning device is configured to be maintained in a fixed position for the duration of the medical procedure.

In certain embodiments, the positioning device is configured to provide positions for Chin lift, Jaw thrust and IV site pressure (to improve IV catheter flows).

In certain embodiments, the extension arm is retractable and is configured to vary in length.

In certain embodiments, the upper support member includes at least one tensioning mechanism.

In certain embodiments, the hand elements are configured to receive a disposable glove.

In certain embodiments, the disposable glove includes a recess, such that a user of the positioning device is able to access the at least one tensioning mechanism in the upper support member through the recess.

In certain embodiments, the hand elements have the shape of a left hand or a right hand.

Other objects of the invention are achieved by providing a patient positioning device for supporting a patient's head, jaw, chin, or neck during a medical procedure, the device comprising: a support member comprising: an upper support member, the upper support member having at least two degrees of freedom, and a lower support member, the lower support member connected to the upper support member and having at least one degree of freedom; an extension arm connected to said at least one of the upper or lower support member; and an attachment member, the attachment member attached at one end to the extension arm and configured to be attached at the other end to a hospital bed, rail or operating room stand.

In certain embodiments, the upper support member has three or more degrees of freedom.

In certain embodiments, the upper support member is movable, such that the upper support member is configured to be locked into a support position and is configured to be unlocked, so as to be movable.

In certain embodiments, the upper support member is configured to be locked into multiple positions. In certain embodiments, the upper support member is configured to be locked into any positions and maintained in such positions.

In certain embodiments, the upper support member comprises at least two fingers, each of the at least two fingers configured to support a patient's head, jaw, chin, or neck.

In certain embodiments, each of the at least two fingers comprise at least one joint, such that each of the at least two fingers have two or more degrees of freedom.

In certain embodiments, each of at least one joints are lockable.

In certain embodiments, the lower support member is connected to the attachment member via a ball and socket joint.

In certain embodiments, the upper support member comprises at least three fingers.

In certain embodiments, the positioning device is configured to be intermittently readjusted during the medical procedure.

In certain embodiments, the positioning device is configured to be maintained in a fixed position for the duration of the medical procedure.

In certain embodiments, the positioning device is configured to provide positions for Chin lift, Jaw thrust and IV site pressure (to improve IV catheter flows).

In certain embodiments, the lower support member is rotatable, such that the upper support member is configured to rotate on said lower support member.

In certain embodiments, the extension arm has two or more degrees of freedom.

In certain embodiments, the extension arm is retractable and is configured to vary in length.

In certain embodiments, the extension arm comprises clamps or retractors.

In certain embodiments, the upper support member comprises and rigid internal structure and an outer padding.

In certain embodiments, the outer padding is soft and/or is made of a material that provides increased comfort to the patient.

In certain embodiments, the upper support member includes at least one tensioning mechanism.

In certain embodiments, the upper support member is configured to receive a disposable glove.

In certain embodiments, the disposable glove includes a recess, such that a user of the positioning device is able to access the at least one tensioning mechanism in the upper support member through the recess.

In certain embodiments, the upper support member has the shape of a left hand or a right hand.

Other objects of the invention are achieved by providing a method for supporting a patient's head, jaw, chin, or neck during a medical procedure, the method comprising: providing a positioning device; supporting the patient's head, jaw, chin, or neck using the positioning device; and readjusting the patient's head, jaw, chin, or neck during the medical procedure.

In certain embodiments, the method involves using a positioning device are described in various embodiments of the invention.

In certain embodiments, the medical procedure involves the administration of anesthesia and/or is a surgical procedure.

In certain embodiments, the patient is rendered unconscious during the medical procedure.

In certain embodiments, the patient may be or is intubated.

Other objects of the invention are achieved by providing a patient positioning device for supporting a patient's head, jaw, chin, or neck during a medical procedure, the device comprising: a support member having at least two degrees of freedom; a first extension arm having a first end and a second end, said first extension arm connected to said support member at the first end; a second extension arm connected to said first extension arm at the second end of the first extension arm; and an attachment member, the attachment member connected to said second extension arm at one end and configured to be attached at the other end to a hospital bed, rail or operating room stand.

In certain embodiments, the positioning device further comprises joints connecting the support member, the first extension arm, the second extension arm and the attachment member.

In certain embodiments, the joints are ball and socket joints, rotary joints, hinge joints, pivot joints, gliding joints, ellipsoidal joints, and saddle joints.

In certain embodiments, said support member has three or more degrees of freedom.

In certain embodiments, said support member is movable, such that said support member is configured to be locked into a support position and is configured to be unlocked, so as to be movable.

In certain embodiments, said support member is configured be locked into multiple positions. In certain embodiments, the multiple positions are precise positions and a variety of positions.

In certain embodiments, said support member comprises at least two fingers, each of the at least two fingers configured to support a patient's head, jaw, chin, or neck.

Other objects of the invention and its features and advantages will become more apparent from consideration of the following drawings and accompanying detailed description. The detailed description and specific examples, while indicating the preferred embodiment of the invention, are intended for purposes of illustration only and are not intended to limit the scope of the invention.

### BRIEF DESCRIPTION OF THE DRAWINGS

FIG. 1 is directed to a patient positioning device according to an embodiment of the present invention.

FIGS. 2A-2C are directed to various features of the patient positioning device of FIG. 1.

FIG. 3A is directed to the patient positioning device of FIG. 1 in a first position.

FIG. 3B is directed to various features of the patient positioning device of FIG. 1 in a second extended position.

FIG. 4 is directed to a patient positioning device of another embodiment of the invention.

FIGS. 5A-5C are directed to various features of the patient positioning device of FIG. 4.

FIG. 6 is directed to various features of the patient positioning device of FIG. 4.

FIGS. 7A-7C are directed to various features of the patient positioning device of FIG. 4.

FIGS. 8A-8B are directed to various features of the patient positioning device of FIG. 4.

FIG. 9 is directed to various features of the patient positioning device of an embodiment of the invention.

FIG. 10 is directed to a patient positioning device of another embodiment of the invention.

### DETAILED DESCRIPTION OF THE INVENTION

In the following description, numerous details are set forth for purpose of explanation. However, one of ordinary skill in the art will realize that the invention may be practiced without the use of these specific details. For instance, the techniques described below are described in a specified order, but other embodiments may change the order of the operations while still embodying the current invention.

As set forth above, the invention is directed to a device that will allow for safe, quick, easy, and consistent maintenance of any one of a variety of positions of a patient during a medical and/or surgical procedure.

In certain embodiments, the device will provide for Chin lift; Jaw thrust; IV site pressure (to improve IV catheter flows) and other such improvements during a medical and/or surgical procedure. In certain embodiments, the device will be used by an anesthesiologist to position the patient's head and/or neck when providing anesthesia to the patient.

In certain positions, the jaw/head neck positioning device is for procedures of the head and/or neck “field avoidance” in which an operator does not readily have access to the sterile surgical field.

The invention provides methods and systems for allowing for easy, quick and possibly continuous, positioning of a patient's body part(s), such as a patient's head and/or neck during a medical and/or surgical procedure.

In certain embodiments, the invention provides for a device that will lock onto the sliding rails on either side of a standard OR table. Apart from the upper portion of the device itself (i.e. the “hand”), the arm of the device will include clamps, retractors, or other devices that allow for the device to rotate and/or have degrees of freedom.

In certain embodiments, the upper portion of the device includes a “hand” that is a metal structure covered in softer padding.

In certain embodiments, the “hand” is made of multiple materials and/or padding, such that the “hand” is firm yet able to be comfortable and to not injure a patient.

In certain embodiments, the “hand” can be wrapped, covered and/or partially covered by a disposable glove to ensure that the “hand” is a hygienic device.

In certain embodiments, these gloves (or other sterile wrap or boundary) are different from conventional gloves in that they have a recess for easy accessibility to the tensioning mechanisms of the hand.

In certain embodiments, the device includes easily accessible tensioning devices that will allow easy unlocking and locking of the digits on the “hand” to provide a comfortable yet sturdy fit to the patient.

In certain embodiments, the hand elements are configured to hold a patient's chin at a mandibular angle, to prevent the backwards motion of the mandible (“retrognathia”). In certain embodiments, the hand elements accomplish this feature during a medical and/or surgical procedure when the patient is being sedated and/or receives anesthesia.

In certain embodiments, the hand elements are configured to create “prognathia”, which is the pulling of the jaw forward with some amount of force.

In certain embodiments, the “hand” elements are provided and hold a patient's chin in place by placing a pressure or tension on the patient's chin, such that the jaw is pressed forward and held into place.

In certain embodiments, both a Right-sided as well as Left-sided “hand” will be available, each providing the same functionality, but from opposite sides (as needed).

In certain embodiments, all or some surfaces which directly contact the patient will have embedded pressure/force sensors with corresponding LED indicators to ensure safe operation of the device.

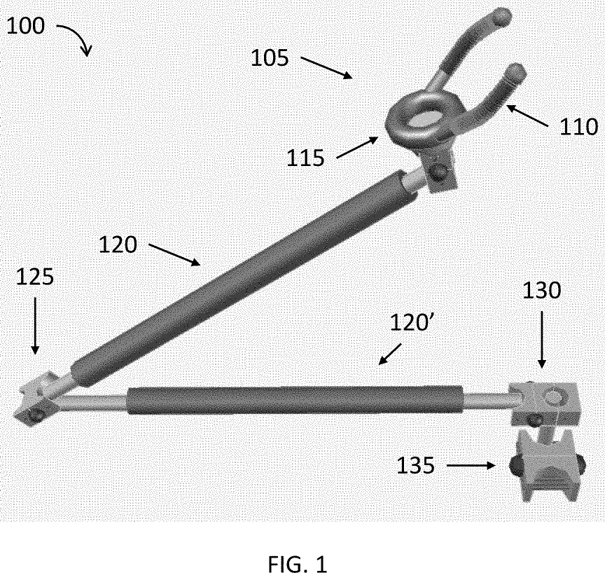

FIG. 1 is directed to an embodiment of the present invention, which shows a patient positioning device (100). In FIG. 1, the patient positioning device includes an upper portion or “hand” element (105). The upper portion (105) includes two flexible jaw supports (110) as well as a chin support (115). In certain embodiments, additional flexible jaw supports are provided as well as additional “fingers” such that a hand shaped portion is provided (Not Shown).

FIG. 1 also includes two telescopic arms (120, 120′) that are adjustable and that are connected via a first rotary joint (125). A second rotary joint (130) is provided which connects the telescopic arm to an end clamp (135).

In certain embodiments, the telescopic arms (120, 120′) are able to slide so as to lengthen or shorten the telescopic arms. In certain embodiments, the telescopic arms have additional joints (Not Shown), so as to be able to rotate in more than two degrees of freedom.

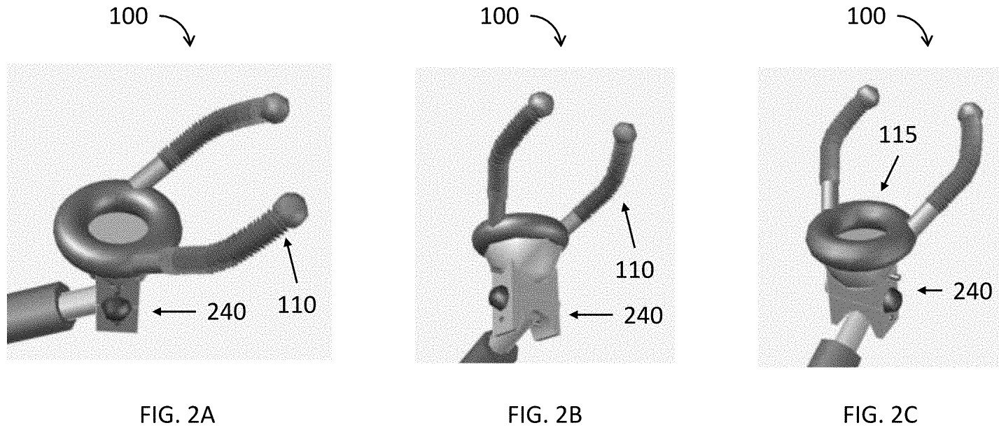

FIGS. 2A-2C provide an exploded view of the upper portion of the device. In FIG. 2A a rotary joint (240) is shown such to provide at least two degrees of freedom to the upper portion of the device (105). In certain embodiments, other types of joints can be provided, such as a ball and socket joint, clamp, or other fastening means that allows the upper portion (105) of the device to rotate with respect to the telescopic arm (120). In certain embodiments, the rotary joint (240) can be fixed in place via a tensioning device or other such securing mechanism.

FIG. 2B is provided to show the flexible jaw supports (110), such that the flexible jaw supports (110) can be used and manipulated such that a person's jaw can be supported by the upper portion of the device (105). In certain embodiments, the rotary joint (240) can be replaced by other types of joints to provide different movement and different degrees of freedom to the device.

FIG. 2C is provided to show the chin support (115) such that a patient's chin can rest in the chin support, while the patient's jaw can rest on the flexible jaw supports to support a patient's head and/or neck. In certain embodiments, the chin support can have different shapes so as to support a chin of a patient. In certain embodiments, the chin support is a platform made of various materials. In certain embodiments, the chin support has a circular or oval shape and is configured to be adapted to the shape of a patient's chin.

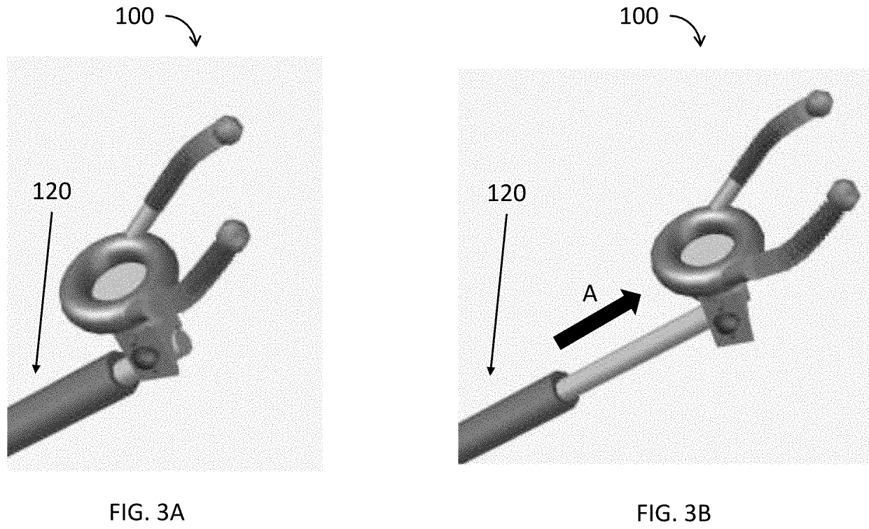

FIG. 3A show the extending telescopic arm (120) in a first position. FIG. 3B shows the extending telescopic arm (120) in a second extended position as depicted by Arrow A. The length of the telescopic arm can be varied depending upon the patient and the position that the patient is to be placed.

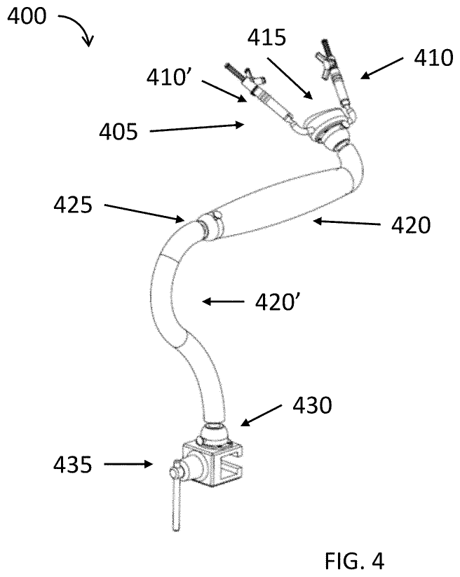

FIG. 4 is directed to an embodiment of the present invention, which shows a patient positioning device (400). In FIG. 4, the patient positioning device includes an upper portion (405). The upper portion (405) includes two flexible jaw supports (410) as well as a chin support (415). In certain embodiments, additional flexible jaw supports are provided as well as additional “fingers” such that a hand shaped portion is provided (Not Shown).

FIG. 4 also includes two arms (420, 420′) that are adjustable similar as to a “goose neck” and that are connected via a first rotary joint (425). A second rotary joint (430) is provided which connects the arm (420′) to an end clamp (435).

In certain embodiments, the two arms (420, 420′) are capable of full articulation when providing adequate rigidity and support in order to place and hold a patient in a desired position. In certain embodiments, the two arms (420, 420′) are capable of being tensioned and being locked into a fixed position.

In certain embodiments, additional strengthening elements or supports are provided in the event a patient's head is large and/or heavy.

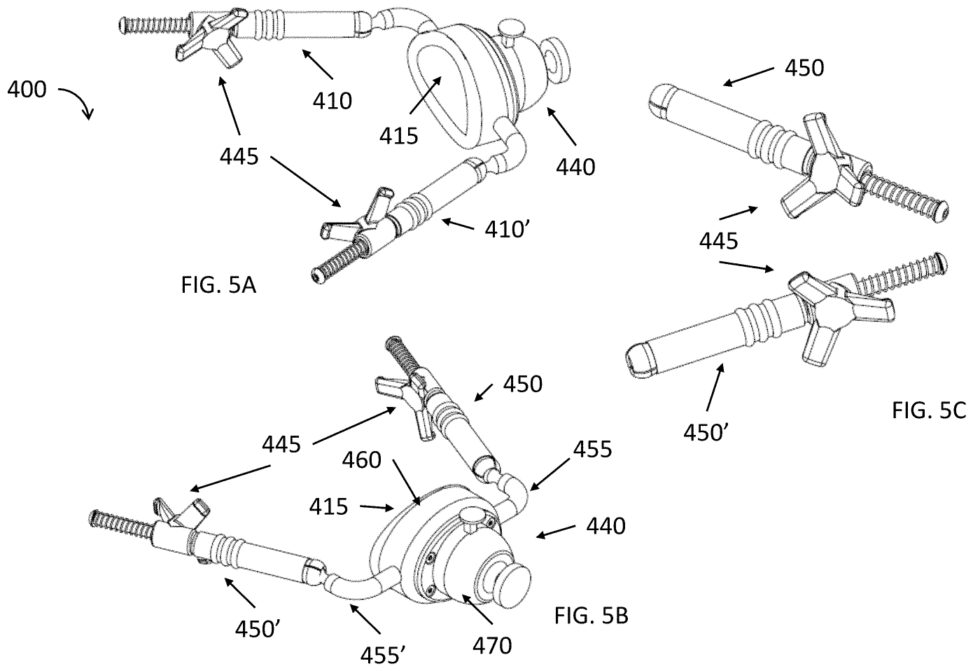

FIGS. 5A-5C depict an embodiment of the present invention (400) as previously described with flexible jaw supports (410, 410′) and including positionable and adjustable mandible pads (445), also referred to as hand elements, for enhanced securing of a patient's jaw and head.

FIG. 5A shows the upper portion (405) having the two flexible jaw supports (410, 410′) as well as a chin support (415). The chin support (415) is configured to have various shapes to support a patient's chin. FIG. 5A shows the hand elements (445) on the two flexible jaw supports (410, 410′). The hand elements (445) are configured to be adjustable to move along the two flexible jaw supports (410, 410′).

In certain embodiments, the hand members are configured to slide along the two flexible jaw supports (410, 410′). In certain embodiments, the hand members are configured to move along the two flexible jaw supports (410, 410′) via an actuation mechanism. In certain embodiments, the hand members are configured to move along the two flexible jaw supports (410, 410′) via rotating the hand elements to cause movement or via a ratcheting mechanism.

As shown in FIGS. 5B and 5C, the two flexible jaw supports (410, 410′) include upper arm members (450, 450′) and lower upper arm members (455, 455′). The upper arm members (450, 450′) are connected to the lower upper arm members (455, 455′) via a ball and socket joint in certain embodiments. In certain embodiments, other types of joints connect the upper arm members (450, 450′) to the lower upper arm members (455, 455′).

In certain embodiments, the upper arm members (450, 450′) are configured to rotate with respect to the lower upper arm members (455, 455′) for easy configuration of the device.

In certain embodiments, the lower upper arm members (455, 455′) have a C-shape and are connected at one end to the housing (460) of the upper portion (405). In certain embodiments, the lower upper arm members (455, 455′) are welded into the housing (460).

As shown in FIG. 5B, the housing (460) of the upper portion (405) is connected to a rotary joint (470), which connects to the extension arm (420). In certain embodiments, the housing (460) is connected to the rotatory joint (470) via one or more fasteners.

As shown in FIG. 5C, the upper arm members (450, 450′) support hand elements (445). In certain embodiments, the hand elements (445) are configured to slide along the upper arm members (450, 450′) in a ratcheted manner. In other embodiments, a push button can be used with an actuator to allow the hand elements (445) to slide or move along the upper arm members (450, 450′) during positioning of the hand elements (445).

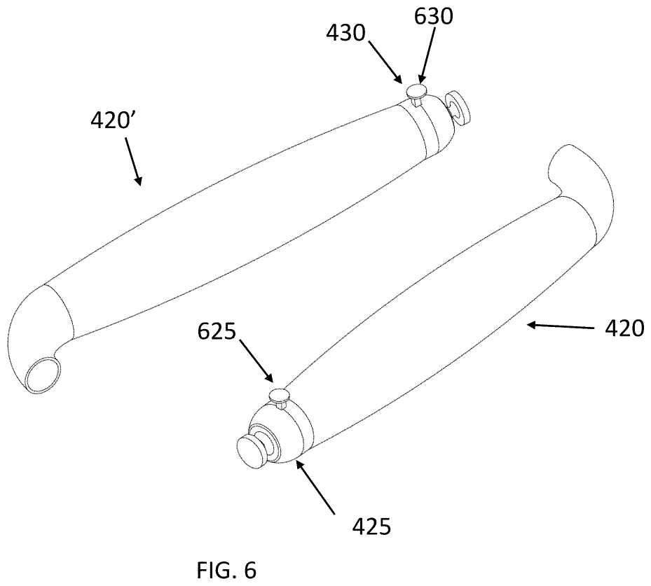

In FIG. 6, two arms (420, 420′) are shown. Arm (420) is connected to first rotary joint (425) which has a stabilizer pin (625). Once the arm is put into the correct configuration, the stabilizer pin (625) can be pressed to secure arm (420) in place. In FIG. 6, arm (420′) is shown connected to rotary joint (430) having stabilizer pin (630).

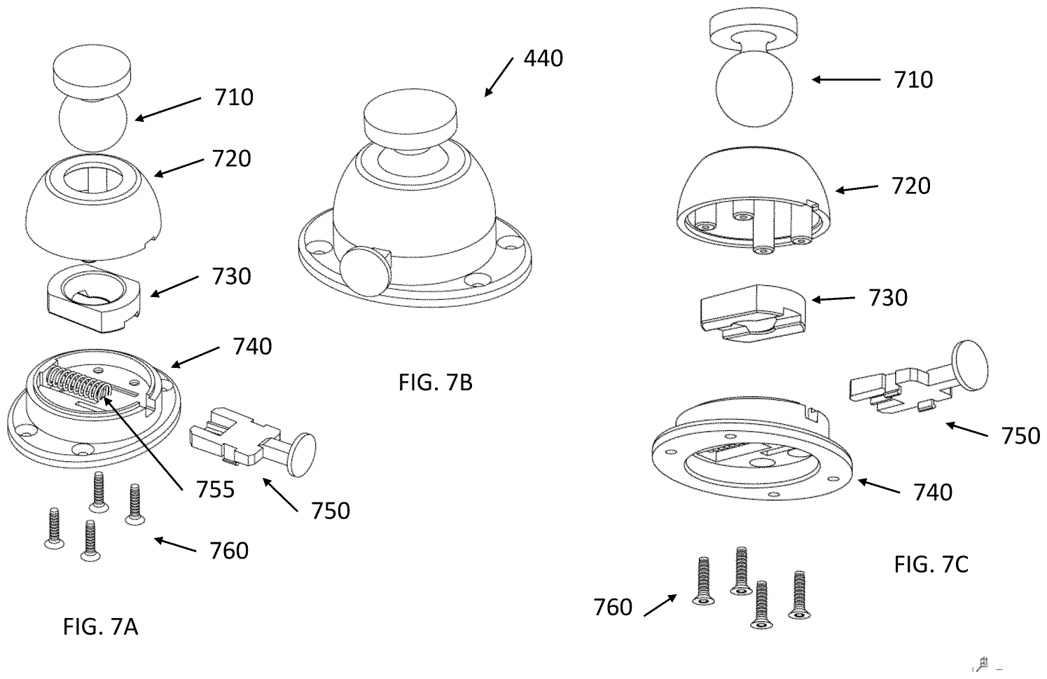

In FIGS. 7A-7C, the components of rotary joint (440) are shown. These components are exemplary and rotary joints having other components are contemplated with regards to this invention. In FIGS. 7A and 7C, the ball joint (710), ball housing (720), ball adapter (730), joint base (740), push-pin (750), spring (755) and fastening means (760) are shown. The ball joint (710) is configured to be received by the ball housing (720) and ball adapter (730) such that the ball joint (710) can rotate within the ball housing (720) and ball adapter (730). This allows for degrees of freedom of rotation of the rotary joint (440). The other rotary joints included in this application are configured to rotate in a similar manner.

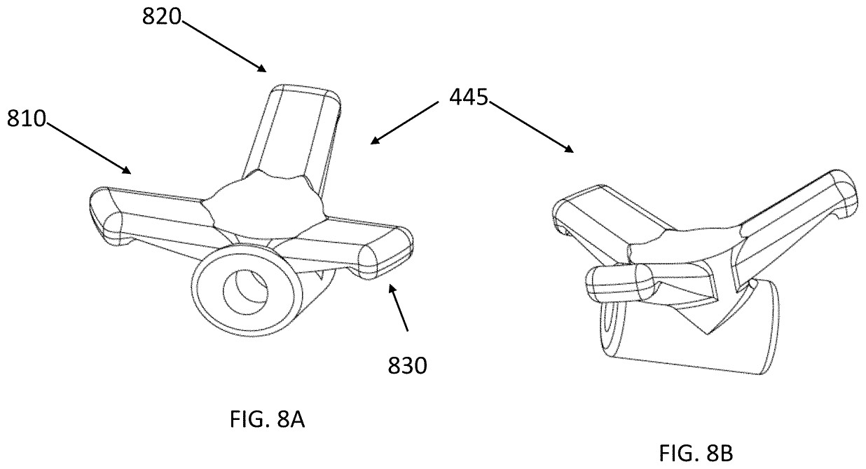

FIG. 8 shows hand elements (445). The hand elements include lug or flange elements (810, 820, 830), which are configured to press against the jaw or head of a person when the positioning device is actuated. The lug or flange elements (810, 820, 830) are ergonomically designed such that they support the jaw or head of a patient with maximum comfort. The lug or flange elements (810, 820, 830) are made of strong materials that can hold the patient's jaw or head in place and when positioned, and a patient's jaw or head can be fixed in place, whereby the jaw is locked in a forward position for assistance with intubation and anesthesia.

In certain embodiments, the hand elements (445) are configured to hold a patient's chin at a mandibular angle, to prevent the backwards motion of the mandible (“retrognathia”) with the effect of sedation.

In certain embodiments, the hand elements (445) are configured to create “prognathia”, which is the pulling of the jaw forward with some amount of force.

In certain embodiments, the hand elements (445) include both right-hand and left-hand elements. In certain embodiments, the lug or flange elements (810, 820, 830) can be individually adjusted to adapt to the curves of a patient's jaw or head.

In certain embodiments, the hand elements can be replaced and/or retrofitted to an individual patient's jaw. In certain embodiments, different side hand elements are used depending upon whether the patient is an adult or child.

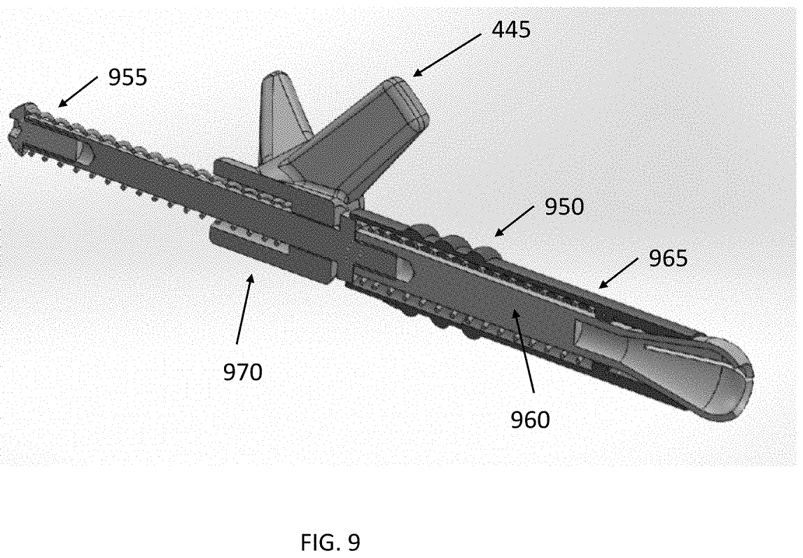

FIG. 9 shows one embodiment of the invention whereby hand element (445) is shown on an upper member (950). In this embodiment, the upper arm member (950) includes ratcheted teeth (955) such that an inner arm (960) slides with an outer arm (965). The hand element (445) is shown having a housing (970) that is configured to adapt to the diameter of the lower arm (960). As the hand element (445) slides, its housing (970) interacts with the ratcheted teeth (955) of the upper arm member (950) and the hand element (445) is able to be manually slide along the upper arm member (950) using a force applied by a user. Once the hand element (445) is placed into the proper configuration, it can be tightened using a tightening member to be secured into place.

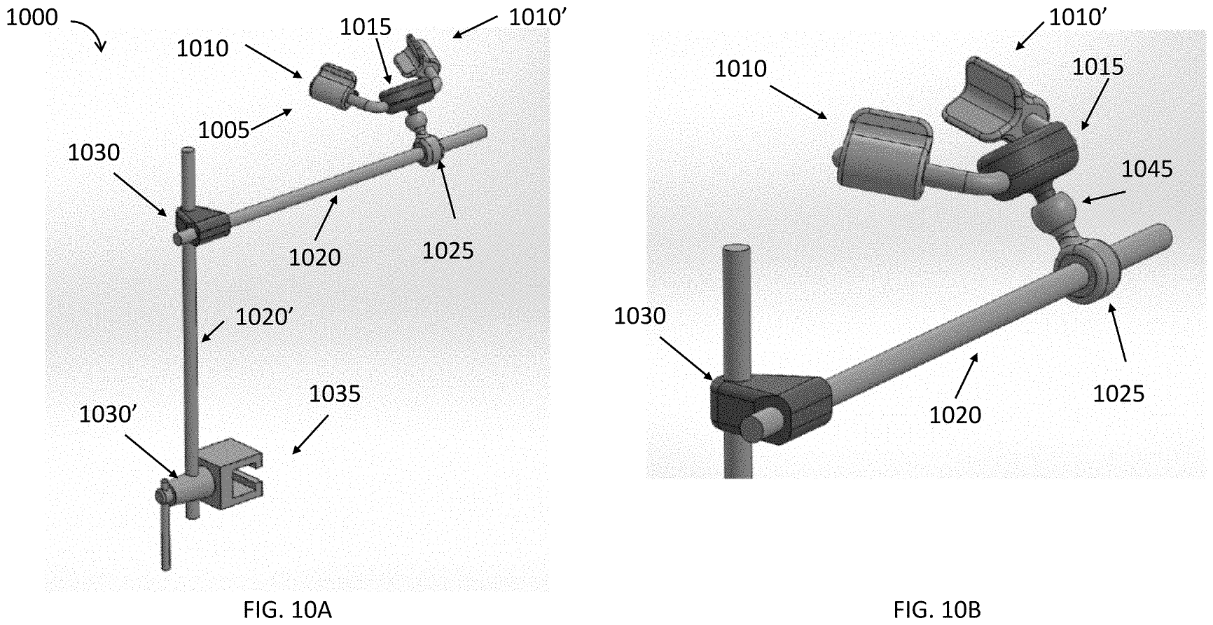

FIGS. 10A and 10B show another embodiment of the patient positioner device (1000). In FIG. 10A, the patient positioning device includes an upper portion (1005). The upper portion (1005) includes two flexible jaw supports (1010, 1010′) as well as a chin support (1015).

FIG. 10A also includes two telescopic arms (1020, 1020′) that are adjustable and that are connected via a tension joint (1030). A second tension (1030′) is provided which connects the telescopic arm (1020′) to an end clamp (1035). In certain embodiments, the telescopic arms (1020, 1020′) are able to slide so move the telescopic arms (1020, 1020′) with respect to the patient.

FIG. 10A also shows the upper portion (1005) configured to rotate with respect to a tension joint (1025). The tension joint (1025) slides along the telescopic arm (1020). As shown in FIG. 10B, the tension joint (1025) is connected to a ball and socket joint (1045), which receives the upper portion (1005). In this regard, the upper portion (1005) is able to rotate with multiple degrees of freedom during positioning of the upper portion (1005).

Upper portion (1005) also includes chin support member (1015) and two flexible jaw supports (1010, 1010′). The flexible jaw supports (1010, 1010′) as shown as having a curved shape with two edges forming a contiguous curve that are able to support a jaw or head of a patient.

In certain embodiments, the flexible jaw supports (1010, 1010′) can be replaced with other elements, for example the hand elements (445) shown in FIGS. 8A and 8B.

While the invention has been specifically described in connection with certain specific embodiments thereof, it is to be understood that this is by way of illustration and not of limitation and that various changes and modifications in form and details may be made thereto, and the scope of the appended claims should be construed as broadly as the prior art will permit.

The description of the invention is merely exemplary in nature, and thus, variations that do not depart from the gist of the invention are intended to be within the scope of the invention. Such variations are not to be regarded as a departure from the spirit and scope of the invention.

1. A patient positioning device for supporting a patient's head, jaw, chin, or neck during a medical procedure, the device comprising:
an upper support member comprising:
a chin support member,
a first jaw support member, and
a second jaw support member,
wherein upper support member includes an upper support member housing for the chin support member, and wherein the first jaw support member and second jaw support member are attached to the housing at one end thereof;
an extension arm having a first end and a second end, said extension arm connected to said upper support member at the first end, wherein the extension arm has at least two degrees of freedom;
an attachment member, the attachment member connected to said extension arm at the second end thereof, said attachment member configured to be attached at the other end to a hospital bed, rail or operating room stand,

2. The positioning device of claim 1, wherein the first jaw support and the second jaw support each includes a hand element.

3. The positioning device of claim 2, wherein the hand elements of each of the first and second jaw support members are configured to be adjustable with respect to the distance from one another and from the chin support member.

4. The positioning device of claim 2, wherein the hand elements are configured to exert a pressure on the patient's chin, wherein the patient's jaw is configured to prevent retraction of the mandible by the hand elements.

5. The positioning device of claim 1, further comprising a rotary joint connecting the attachment member to the extension arm and the extension arm to the upper support member.

6. The positioning device of claim 1, wherein the first jaw support member and the second jaw support are flexible and are configured to rotate with respect to the chin support member.

7. The positioning device of claim 1, wherein the first jaw support member includes a first upper arm member and a first lower arm member, and wherein the second jaw support member includes a second upper arm member and a second lower arm member, wherein the first lower arm member and the second lower arm member are each connected to the upper support member housing.

8. The positioning device of claim 7, wherein the first lower arm member and the second lower arm member are curved and have a C-shape orientation.

9. The positioning device of claim 7, wherein the first upper arm member and the second upper arm member are rods, wherein the first upper arm member is connected to the first lower arm member via a ball and socket joint, and wherein the second upper arm member is connected to the second lower arm member via a ball and socket joint.

10. The positioning device of claim 8, wherein the hand elements are configured to be adjusted along the length rods of the first upper arm member and second upper arm member respectively.

11. The positioning device of claim 1, wherein the chin support member includes a platform for supporting the chin of a patient, wherein the platform is wide enough to support a patient's chin having various sizes.

12. The positioning device of claim 1, wherein the extension member comprises a first extension member and a second extension member that are connected via a first rotary joint.

13. The positioning device of claim 1, wherein the first extension member and the second extension member are configured to provide full articulation when providing adequate rigidity and support in order to place and hold a patient in a desired position.

14. The positioning device of claim 1, further comprising a pressure force sensor embedded in the chin support member.

15. The positioning device of claim 14, further comprising a display embedded within the positioning device, the display showing the colors red, yellow and green to associate different locking positions of the device in order to optimize both the precise position and forces applied to the device.

16. (canceled)

17. The positioning device of claim 1, wherein the extension member is configured to be locked during support of the patient and unlocked during manipulation of the positioning device prior to positioning the patient.

18. The positioning device of claim 1, wherein the positioning device is configured to be intermittently readjusted during the medical procedure.

19. The positioning device claim 1, wherein the positioning device is configured to be maintained in a fixed position for the duration of the medical procedure.

20. The positioning device claim 1 to 19, wherein the positioning device is configured to provide positions for Chin lift, Jaw thrust and IV site pressure (to improve IV catheter flows).

21. (canceled)

22. The positioning device of claim 1, wherein the upper support member includes at least one tensioning mechanism.

23. The positioning device of claim 2, wherein the hand elements are configured to receive a disposable glove to provide an extra barrier protection, wherein the disposable glove includes a recess, such that a user of the positioning device is able to access the at least one tensioning mechanism in the upper support member through the recess.

24.-60. (canceled)
